(19) United States
(12) Patent Application Publication
(10) Pub. No.: US 2007/0219426 A1
(43) Pub. Date: Sep. 20, 2007

(54) Subscription-Based Private Civil Security Resource Customization Method

(76) Inventor: Barrett H. Moore, Winnetka, IL (US)

Correspondence Address: FITCH EVEN TABIN AND FLANNERY, 120 SOUTH LA SALLE STREET, SUITE 1600, CHICAGO, IL, 60603-3406, US

(21) Appl. No.: 11/466,727

(22) Filed: Aug. 23, 2006

Related U.S. Application Data
(63) Continuation-in-part of application No. 11/384,037, filed on Mar. 17, 2006.
Application No. 11/384,037 is a continuation-in-part of application No. 11/394,350, filed on Mar. 30, 2006.
Application No. 11/394,350 is a continuation-in-part of application No. 11/279,333, filed on Apr. 11, 2006.
Application No. 11/279,333 is a continuation-in-part of application No. 11/379,929, filed on Apr. 24, 2006.
Application No. 11/379,929 is a continuation-in-part of application No. 11/381,247, filed on May 2, 2006.
Application No. 11/381,247 is a continuation-in-part of application No. 11/381,257, filed on May 2, 2006.
Application No. 11/381,257 is a continuation-in-part of application No. 11/381,265, filed on May 2, 2006.
Application No. 11/381,265 is a continuation-in-part of application No. 11/381,265, filed on May 2, 2006.
Application No. 11/381,265 is a continuation-in-part of application No. 11/381,277, filed on May 2, 2006.
Application No. 11/381,277 is a continuation-in-part of application No. 11/383,022, filed on May 12, 2006.
Application No. 11/383,022 is a continuation-in-part of application No. 11/420,594, filed on May 26, 2006.
Application No. 11/420,594 is a continuation-in-part of application No. 11/421,694, filed on Jun. 1, 2006.
Application No. 11/421,694 is a continuation-in-part of application No. 11/423,594, filed on Jun. 12, 2006.
Application No. 11/423,594 is a continuation-in-part of application No. 11/425,043, filed on Jun. 19, 2006.
Application No. 11/425,043 is a continuation-in-part of application No. 11/426,231, filed on Jun. 23, 2006.
Application No. 11/426,231 is a continuation-in-part of application No. 11/456,472, filed on Jul. 10, 2006.
Application No. 11/456,472 is a continuation-in-part of application No. 11/461,605, filed on Aug. 1, 2006.
Application No. 11/461,605 is a continuation-in-part of application No. 11/461,624, filed on Aug. 1, 2006.
Application No. 11/461,624 is a continuation-in-part of application No. 11/462,795, filed on Aug. 7, 2006.
Application No. 11/462,795 is a continuation-in-part of application No. 11/462,845, filed on Aug. 7, 2006.
Application No. 11/462,845 is a continuation-in-part of application No. 11/464,751, filed on Aug. 15, 2006.
Application No. 11/464,751 is a continuation-in-part of application No. 11/464,764, filed on Aug. 15, 2006.
Application No. 11/464,764 is a continuation-in-part of application No. 11/464,775, filed on Aug. 15, 2006.
Application No. 11/464,775 is a continuation-in-part of application No. 11/464,788, filed on Aug. 15, 2006.
Application No. 11/464,788 is a continuation-in-part of application No. 11/464,799, filed on Aug. 15, 2006.
Application No. 11/464,799 is a continuation-in-part of application No. 11/465,063, filed on Aug. 16, 2006.

(51) Int. Cl. A61B 5/00

(52) U.S. Cl. 600/300

(57) ABSTRACT

A private civil security subscription mechanism serves to facilitate the provision of customized life-sustaining resources for corresponding authorized beneficiaries. The customization of these resources may be the based on the needs, requirements, or preferences of the subscriber, authorized beneficiary, expert, and/or another third party. The life-sustaining resources may or may not be delivered with additional non-necessity resources. A personal profile may be created to track the characterizing attributes, needs, requirements, and preferences of the authorized beneficiary and may also store information regarding the customized resources.

### RELATED APPLICATIONS

This comprises a continuation-in-part of each of:

SUBSCRIPTION-BASED PRIVATE CIVIL SECURITY FACILITATION METHOD as filed on Mar. 17, 2006 and having application Ser. No. 11/384,037;

SUBSCRIPTION-BASED CATASTROPHE-TRIGGERED MEDICAL SERVICES FACILITATION METHOD as filed on Mar. 30, 2006 and having application Ser. No. 11/394,350;

PERSONAL PROFILE-BASED PRIVATE CIVIL SECURITY SUBSCRIPTION METHOD as filed on Apr. 11, 2006 and having application Ser. No. 11/279,333;

RADIATION SHELTER KIT APPARATUS AND METHOD as filed on Apr. 24, 2006 and having application Ser. No. 11/379,929;

FRACTIONALLY-POSSESSED UNDERGROUND SHELTER METHOD AND APPARATUS as filed on May 2, 2006 and having application Ser. No. 11/381,247;

SUBSCRIPTION-BASED CATASTROPHE-TRIGGERED TRANSPORT SERVICES FACILITATION METHOD AND APPARATUS as filed on May 2, 2006 and having application Ser. No. 11/381,257;

SUBSCRIPTION-BASED MULTI-PERSON EMERGENCY SHELTER METHOD as filed on May 2, 2006 and having application Ser. No. 11/381,265; and

SUBSCRIPTION-BASED CATASTROPHE-TRIGGERED RESCUE SERVICES FACILITATION METHOD AND APPARATUS as filed on May 2, 2006 and having application Ser. No. 11/381,277;

DOCUMENT-BASED CIVILLY-CATASTROPHIC EVENT PERSONAL ACTION GUIDE FACILITATION METHOD as filed on May 12, 2006 and having application Ser. No. 11/383,022;

RESCUE CONTAINER METHOD AND APPARATUS as filed on May 26, 2006 and having application Ser. No. 11/420,594;

PURCHASE OPTION-BASED EMERGENCY SUPPLIES PROVISIONING METHOD as filed on Jun. 1, 2006 and having application Ser. No. 11/421,694;

SUBSCRIPTION-BASED PRE-PROVISIONED TOWABLE UNIT FACILITATION METHOD as filed on Jun. 12, 2006 and having application Ser. No. 11/423,594;

RADIATION-BLOCKING BLADDER APPARATUS AND METHOD as filed on Jun. 19, 2006 and having application Ser. No. 11/425,043; and

PRIVATE CIVIL DEFENSE-THEMED TELEVISION BROADCASTING METHOD as filed on Jun. 23, 2006 and having application Ser. No. 11/426,231;

EMERGENCY SUPPLIES PRE-POSITIONING AND ACCESS CONTROL METHOD as filed on Jul. 10, 2006 and having application Ser. No. 11/456,472;

PRIVATE CIVIL DEFENSE-THEMED BROADCASTING METHOD as filed on Aug. 1, 2006 and having application Ser. No. 11/461,605; and

METHOD OF PROVIDING VARIABLE SUBSCRIPTION-BASED ACCESS TO AN EMERGENCY SHELTER as filed on Aug. 1, 2006 and having application Ser. No. 11/461,624;

SUBSCRIPTION-BASED INTERMEDIATE SHORT-TERM EMERGENCY SHELTER METHOD as filed on Aug. 7, 2006 and having application Ser. No. 11/462,795;

SUBSCRIPTION-BASED CATASTROPHE-TRIGGERED RESCUE SERVICES FACILITATION METHOD USING WIRELESS LOCATION INFORMATION as filed on Aug. 7, 2006 and having application Ser. No. 11/462,845;

PRIVATELY PROVISIONED SURVIVAL SUPPLIES DELIVERY METHOD as filed on Aug. 15, 2006 and having application Ser. No. 11/464;751;

PRIVATELY PROVISIONED SURVIVAL SUPPLIES SUB-UNIT-BASED DELIVERY METHOD as filed on Aug. 15, 2006 and having application Ser. No. 11/464,764;

PRIVATELY PROVISIONED SURVIVAL SUPPLIES ACQUISITION METHOD as filed on Aug. 15, 2006 and having application Ser. No. 11/464,775;

PRIVATELY PROVISIONED SURVIVAL SUPPLIES CONTENT ACQUISITION METHOD as filed on Aug. 15, 2006 and having application Ser. No. 11/464,788;

METHOD TO PRIVATELY PROVISION SURVIVAL SUPPLIES THAT INCLUDE THIRD PARTY ITEMS as filed on Aug. 15, 2006 and having application Ser. No. 11/464,799;

WASTE DISPOSAL DEVICE as filed on Aug. 16, 2006 and having application Ser. No. 11/465,063;

the contents of each of which are fully incorporated herein by this reference.

### TECHNICAL FIELD

This invention relates generally to the provision of life-sustaining resources.

### BACKGROUND

Many citizens of the world have long passed the point when a ready availability of the basic necessities of life is satisfactory in and of itself. Today's consumer-oriented citizens demand, and often receive, an incredibly diverse and seemingly ever-growing cornucopia of consuming and experiential options. Such riches are typically based, in turn, upon a highly interdependent series of foundational infrastructure elements.

Examples of the latter include, but are certainly not limited to:

transportation infrastructure such as roads, bridges, railways, and so forth that facilitate the inexpensive and rapid movement of sometimes perishable goods from source to consumer;

communications infrastructure such as telephones, television, radio, and the Internet that facilitate the inexpensive and rapid sharing of news, advice, information, and entertainment; and

the totality of civil services such as police services, fire fighting services, medical services, and so forth that facilitate a sufficient degree of order and predictability to, in turn, permit the complex series of inter-related actions that modern society requires in order to operate.

As powerful as the machinery of modern life appears, however, modern citizens are today perhaps more at risk of experiencing a serious disruption in their ability to prosper or even to survive en mass than is generally perceived. Providing the necessities of life in general requires a lot of things to all operate, more or less, correctly. To put it another way, a serious disruption to any significant element of civilized infrastructure can produce catastrophic results for a broad swath of a given civil community. Any number of natural and/or non-naturally-caused events can greatly disrupt society's infrastructure and ability to provide one or more life-sustaining resources such as water, nutrition, shelter, and the like.

This situation exists in large measure due to the just-in-time nature of modern inventory and control schemes and practices. As but one example, studies have shown that a typical modern urban grocery store has but a few days worth of inventory on hand at any given time. Without a virtually constant re-supply stream, shelves would quickly go bare. A significant disruption to supply chains, then, can lead to a rapid depletion of available stock. This, in turn, can lead to critical shortages of necessary emergency supplies at the very moment when such supplies are most needed. The unpredictability with respect to what supplies may become limited in this regard can comprise a particularly troubling component of this problem.

Many people believe and trust that their government (local, regional, and/or national) will provide for them in the event of such a civilly-catastrophic event. And, indeed, in the long view such is clearly a legitimate responsibility owed by any government to its citizens. That such is a consummation devoutly to be wished, however, does not necessarily make it so. Hurricane Katrina provided some insight into just how unprepared a series of tiered modern governmental entities may actually be to respond to even basic survival needs when a catastrophic event occurs.

Such insights, of course, are not particularly new. Civil preparedness shortcomings occasionally attract public attention and niche marketing opportunities exist with respect to provisioning the needs of so-called survivalists. Indeed, there are those who spend a considerable amount of their time and monetary resources attempting to ready themselves to personally survive a civilly-catastrophic event. Therein, however, lies something of a conundrum.

On the one hand, modern governments typically do little to proactively ensure the bulk provisioning (let alone the comfort) of their citizens in the face of many or most catastrophic events. On the other hand, attempting to take responsible actions to reasonably ensure one's own safety and security can become, in and of itself, nearly a full-time avocation and leave little time to actually enjoy the conveniences and opportunities of modern life. Such individual actions may even be frowned upon by the greater part of society which has grown accustomed to and falsely secure with existing efficient just-in-time delivery systems that provide the illusion of plenty while undercutting the perception of risk.

As a result, many (if not most) individuals and their families are largely bereft of the supplies that they will need should a civilly-catastrophic event befall them. This shortcoming tends to be relatively comprehensive; most people have neither a sufficient selection of survival supplies nor a sufficient quantity. For people who do have a store of supplies set aside against such an eventuality, it can be a considerable burden to maintain and ensure the freshness, vitality, and usability of those supplies. At the same time, the same civilly-catastrophic event that occasions their need for supplies will also likely disrupt relevant supply chains enough to cause a partial or complete shortage of supplies at their local merchants. The unfortunate net result is a relatively near term severe need for a variety of survival supplies that will often go unmet for lengthy periods of time.

### BRIEF DESCRIPTION OF THE DRAWINGS

The above needs are at least partially met through provision of the Subscription-based Private Civil Security Resource Customization Method described in the following detailed description, particularly when studied in conjunction with the drawings, wherein:

FIG. 1 comprises a flow diagram as configured in accordance with various embodiments of the invention.

Skilled artisans will appreciate that elements in the figure are illustrated for simplicity and clarity. For example, common but well-understood elements that are useful or necessary in a commercially feasible embodiment are often not described in order to facilitate a less obstructed understanding of these various embodiments of the present invention. It will further be appreciated that certain actions and/or steps may be described or depicted in a particular order of occurrence while those skilled in the art will understand that such specificity with respect to sequence is not actually required. It will also be understood that the terms and expressions used herein have the ordinary meaning as is accorded to such terms and expressions with respect to their corresponding respective areas of inquiry and study except where specific meanings have otherwise been set forth herein.

### DETAILED DESCRIPTION

Generally speaking, pursuant to these various embodiments, a subscription-based approach serves to facilitate the procurement of life-sustaining resources for corresponding authorized beneficiaries and the customization of such life-sustaining resources. Customization of the at least one life-sustaining resource may be based upon the authorized beneficiary's needs, requirements, or preferences. In this manner, the authorized beneficiary is provided access to the life-sustaining resource of the beneficiary's choice. Alternatively, the life-sustaining resource may be customized for the authorized beneficiary by another such as by an expert or guardian. Personal information, such as the authorized beneficiary's characterizing attributes and preferences, may be stored in a personal profile that can be updated periodically. The life-sustaining resource may include a plurality of life-sustaining resources and the authorized beneficiaries may be provided with an additional non-necessity resource. Further, by one approach, the resources are procured and maintained pending a need to permit subscription-based access to the resource(s) in the event of a catastrophic event.

These steps are readily facilitated without dependency upon governmental oversight, participation, or control. The particular resources and/or quantity of resources provided can vary with the needs and requirements of the authorized beneficiaries. Importantly, via these teachings, an individual can take important steps to bring a considerably improved measure of security into their lives without having to effectively become a full-time survivalist; such individuals can, in short, continue to enjoy their chosen vocations and standard of living knowing that, should a civilly-catastrophic event indeed be visited upon them, they will have extraordinary access to one or more resources that are critical to human life.

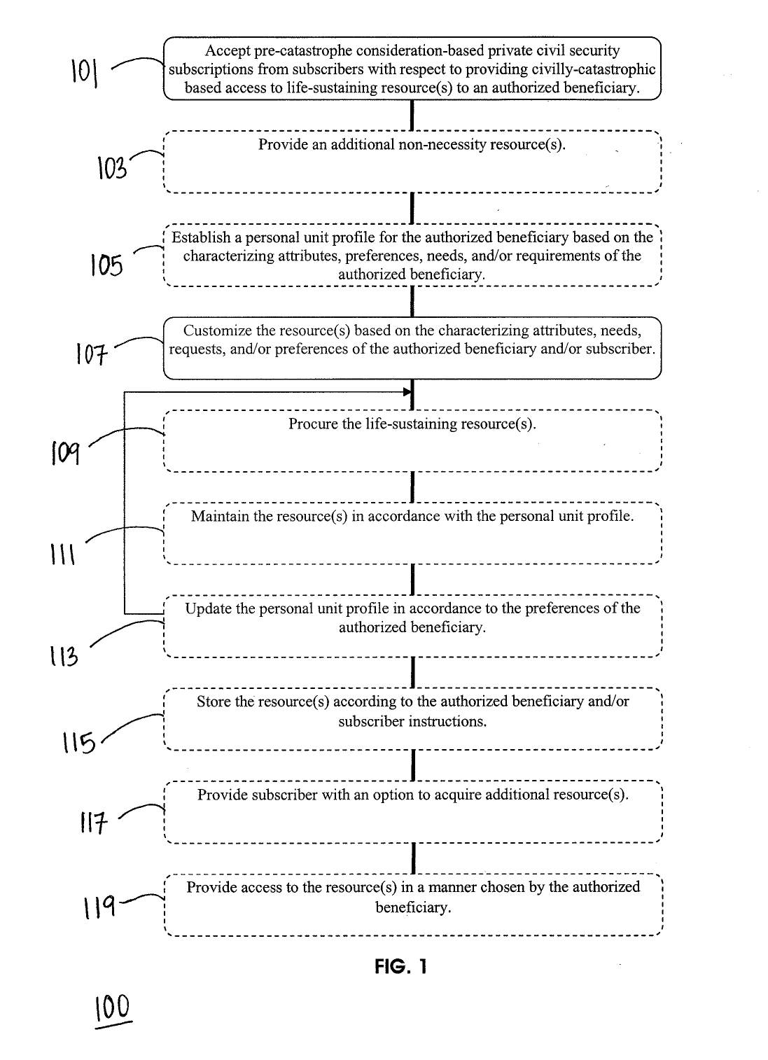

These and other benefits may become clearer upon making a thorough review and study of the following detailed description. Referring now to the drawings, and in particular to FIG. 1, an illustrative process 100 provides for accepting 101 consideration-based private civil security subscriptions from corresponding subscribers with respect to providing civilly-catastrophic event-based access to at least one customized life-sustaining resource. This right of access to the life-sustaining resource can pertain, if desired, to a predetermined quantity of the life-sustaining resource or a predetermined time-frame. For example, a given subscription can relate to providing access to a one year's supply of at least one life-sustaining resource to one or more authorized beneficiaries.

By one approach, these subscriptions may be accepted by, for example, a for-profit business. By another approach a not-for-profit business (such as a membership-based entity) may be the appropriate entity to offer and accept such subscriptions. Such subscriptions provide access to one or more life-sustaining resources (such as one or more selections from one or more of life's necessities such as hydration supplies, nourishment supplies, environmentally-borne threat abatement supplies, and/or medical supplies).

These teachings also readily encompass the notion of a given subscriber providing such a subscription for an authorized beneficiary other than themselves. Such might occur, for example, when one family member procures such a subscription for one or more other family members. Another example would be for a company to subscribe on behalf of named key employees, family members of such key employees, and so forth. Other examples no doubt exist.

As noted these teachings provide for a subscription-based approach. As used herein, the term “subscription” shall be understood to refer to and encompass a variety of legal mechanisms. Some relevant examples include, but these teachings are not limited to, subscription mechanisms such as:

time-limited rights of access (as where a subscription provides access rights for a specific period of time, such as one year, in exchange for a corresponding series of payments);

event-limited rights of access (as where a subscription provides access rights during the life of a given subscriber based upon an up-front payment in full and where those access rights terminate upon the death of the subscriber or where, for example, a company purchases a subscription for a key employee and those corresponding rights of access terminate when and if that key employee leaves the employment of that company);

inheritable rights of access (as may occur when the subscription, by its own terms and conditions, provides a right of access that extend past the death of a named subscription beneficiary and further provides for testate and/or intestate transfer to an heir);

rights of access predicated upon a series of periodic payments (as where a subscription provides access rights during, for example, predetermined periods of time on a periodic basis as where a subscriber offers a payment on a month-by-month bases to gain corresponding month-by-month access rights);

ownership-based rights of access (as may occur when the subscription provides for ownership rights that pertain to the life-sustaining resources);

non-transferable rights of access (as may occur when the subscription by its terms and conditions, prohibits transfer of the right of access from a first named beneficiary to another);

transferable rights of access (as may occur when the subscription, by its terms and conditions, permits, conditional or unconditional transfer of the right of access from a first named beneficiary to another);

membership-based rights of access (as may occur when the subscription, by its terms and conditions, establishes a membership interest with respect to the accorded rights of access, such as, for example, a club-based membership);

fractionally-based rights of access (as may occur when the subscription, by its terms and conditions, establishes a divided or undivided co-ownership interest by and between multiple subscription beneficiaries with respect to a right to access customized life-sustaining resources);

non-ownership-based rights of access (as may occur when the subscription, by its terms and conditions, establishes the aforementioned right of access via, for example, a least, a rental, or borrowing construct); and/or option-based rights of access (as may occur when the subscription, by its terms and conditions, provides an opportunity to acquire a future right of access based upon some additional event such as an additional offering of consideration).

As noted, these subscriptions relate to providing access to one or more life-sustaining resources in the event of a civilly-catastrophic event. Such services may be predicated, if desired, upon a requirement that the civilly-catastrophic event be one that lasts for a predetermined period of time (such as an hour, one day, one week, or so forth) or that causes at least a predetermined amount or degree of civil infrastructure impairment or other measurable impact of choice.

As used herein, “civilly-catastrophic event” will be understood to refer to an event that substantially and materially disrupts a society's local, regional, and/or national infrastructure and ability to provide in ordinary course for the survival needs of an impacted community. Such a civilly-catastrophic event can include both a precipitating even (which may occur over a relatively compressed period of time or which may draw out over an extended period of time) as well as the resultant aftermath of consequences wherein the precipitating event and/or the resultant aftermath include both the cause of the infrastructure interruption as well as the continuation of that interruption.

A civilly-catastrophic event can be occasioned by any of a wide variety of natural and/or non-naturally-cased disasters. Examples of natural disasters that are potentially capable of initiating a civilly-catastrophic event include, but are not limited to, extreme weather-related events (such as hurricanes, tsunamis, extreme droughts, widespread or unfortunately-targeted tornadoes, extreme hail or rain, and the like, flooding, and so forth), extreme geological events (such as earthquakes, volcanic activity, and so forth) severe astronomically-based events (such as extreme solar flares, collisions with comets or large asteroids, and so forth), extreme environmental events (such as widespread uncontrolled fire or the like), severe disease-based events such as global or regional pandemics, a severe natural shortage of a life-sustaining resource, and so forth, to note but a few.

Examples of non-naturally occurring disasters capable of initiating a civilly-catastrophic event include both unintended events as well as acts of war, terrorism, madness or the like. Examples of non-naturally-caused disasters capable of such potential scale include, but are not limited to, nuclear-related events (including uncontrolled fission or fusion releases, radiation exposure, and so forth), acts of war, terrorism, and related aggression, the accidental or intentional release and public dispersal of a severe environmentally borne danger such as deadly or otherwise disruptive biological or chemical agents or creations, and so forth.

This process permits authorized beneficiaries and subscribers to customize 107 the life-sustaining resources. Accordingly, a plurality of differentiated subscription opportunities are offered. Additionally (or in lieu of such an approach), the resource provider or an interested third party may customize the resources for the authorized beneficiary. As yet another example, the subscriber may seek to customize the resources provided on behalf of a given authorized beneficiary. This plurality of differentiated subscription opportunities can correspond, for example, to providing access to different categories, quantities, types, or qualities of resources and different bundles or groups of resources. As but one very simple illustration in this regard, such subscription opportunities can differ from one another at least with respect to cost. This, in turn, provides subscriber choice with respect to selecting a particular subscription that best meets their specific needs, preferences, and/or budget limitations. For example, one subscription can provide for accessing life-sustaining resources that, though nutritionally viable, are economically selected while another subscription might provide for life-sustaining resources that are more costly and in turn reflect, for example, a wider variety of choices within a given category of resource. As yet another example, the subscriptions may differ by gender or age such that the life-sustaining resources provided may be tailored to meet the various recommended nutrition intake for a particular age group or gender. Alternatively, the average preferences or typical choices of a certain demographic may be used to provide differentiated subscriptions tailored to one's likely preferences, much in the same way a children's menu caters to youthful food preferences and recommended serving sizes.

In one form, a plurality of life sustaining resources are provided such that the average authorized beneficiary will be able to sustain life for a certain period of time, such as eighteen months. It is contemplated that a standard unit of staple items including the most basic life sustaining resources will be available. While some beneficiaries may chose to receive only the basic supplies, other may chose to tailor the resources received. By one approach, such a standard unit can comprise the foundation or basic building block of the unit eventually provided for particular authorized beneficiaries. Substitutions, alterations, and/or deletions to this basic unit will effect a change to the specific resources that a given authorized beneficiary will be provided. These teachings will accommodate an authorized beneficiary who requests so many upgrades that not a single item in the standard unit will be received by the authorized beneficiary. By another approach, the life-sustaining resources are chosen individually or in an a la carte fashion such that an authorized beneficiary may only choose to have access to a few particular items for a certain period of time.

The life-sustaining resources provided may be a consumable necessity of human life or a non-consumable necessity of human life. In this regard, hydration supplies are an example of a consumable necessity of human life. Further, for some, medicine taken on a daily basis may be a consumable necessity of human life, while certain medical devices may be a non-consumable necessity for human life. An illustrative listing of representative necessities comprises, but is certainly not limited to:
hydration supplies (including potable water, water purification chemicals, sports drinks, energy drinks, and so forth as well as non-consumable items such as a water purification apparatus, and so forth);
nourishment supplies (including foods of various kinds such as fresh, canned, freeze-dried, frozen, refrigerated, raw, and processed food, vitamin supplements, and so forth);
medical supplies (including consumable items such as dressings, medicines, vaccines, blood, plasma products, and so forth, as well as non-consumable items such as ventilators, and so forth);
environmentally borne threat abatement supplies and personal protection supplies (breathable air supplies, sanitation supplies, electromagnetic pulse recovery supplies including but not limited to spare vehicular electronic control modules, face masks, gas masks, head gear such as protective hats or helmets, ionizing radiation resistant coveralls, wearable dosimeters, self defense weaponry, and so forth);
   to illustrate but a few examples.

In regard to the consumable necessities, a seemingly endless list is available to fulfill life's daily nutritional requirements. Any edible item having calories, vitamins, nutrients, and/or minerals may fall within the definition of nourishment supplies. Customizing the nourishment supplies received by an authorized beneficiary allows the beneficiary to choose nourishment supplies to suit the beneficiary's physical needs or preferences. By one approach, an authorized beneficiary may choose to have strictly vegetarian or vegan nourishment supplies. Other authorized beneficiaries may chose to only have kosher nourishment supplies. In a similar manner, authorized beneficiaries may choose different types of supplies. For example, with respect to hydration supplies, one authorized beneficiary may prefer to receive bottled water, while another may prefer a particular sports drink.

A food allergy or a food intolerance prevents or discourages some individuals from eating certain types or categories of foods. The result from eating these foods can vary from a mild unpleasant reaction to a severe adverse reaction triggered by the immune system. In some cases, a hypersensitivity to certain foods can even cause a life-threatening allergic reaction (anaphylaxis) that can partially or wholly constrict airways, severely lower blood pressure, and cause suffocation by the swelling of the tongue or throat. The most common food allergies are slightly different for children and adults. Common food allergens include, but are certainly not limited to: shellfish, such as shrimp, crayfish, lobster, and crab; peanuts; tree nuts such as walnuts; fish; corn; milk (dairy); soy; sugar; wheat; yeast; and eggs. Thus, it is contemplated that an authorized beneficiary may desire to choose nourishment supplies based on excluding certain types or categories of foods. In addition, due to the extremely sensitive nature of some allergies, some authorized beneficiaries may request that the nourishment supplies to be received are not initially prepared, containerized, stored, or otherwise brought into contact with certain other foods or categories of foods.

The quality of life-sustaining resources may vary by subscription. For example, not only may an authorized beneficiary prefer bottled water to any other type of hydration supply, the beneficiary may specify a particular brand of bottled water to be provided. An authorized beneficiary may chose to upgrade all of the nourishment supplies such that the authorized beneficiary receives only luxury, top-shelf, or brand name life-sustaining resources. While delicacies and other luxury food items, such as caviar, are not typically categorized as a necessity, these food items do provide nourishment required to sustain human life and therefore may be viewed as a life-sustaining resource and consumable necessity in the sense that these resources provide sustenance.

The quality of a life-sustaining resource may also include the specific handling or storage of particular resources to be received. By one approach, the authorized beneficiary may choose to only receive organic or free-range nourishment supplies. In another example, a particular nourishment supply may be best consumed in a particular six month period. If an authorized beneficiary chooses to request that particular nourishment supply, the authorized beneficiary may also outline supply specifics such as how often the resource should be updated or from where the resource must be acquired.

As one option, if desired, this process 100 can further accommodate providing 103 an additional non-necessity resource (that is, an item that is not, strictly speaking, necessary to sustain human life on an elemental basis). This can certainly comprise providing civilly-catastrophic event-based access to a plurality of non-necessities if desired. As suggested above with respect to the necessities, an almost infinite number of possibilities are available to be chosen. An illustrative listing of representative non-necessities comprises, but is certainly not limited to:
clothing (such as a coat, a jacket, a poncho, a top, a sweater, pants, trousers, jeans, shorts, belts, undergarments, socks, footwear such as sandals, boots, shoes, sportswear, a skirt, a dress, swimwear, head gear, cold-weather wear including, but not limited to, hats, gloves, scarves, and so forth);
sundries (such as batteries, flash lights, matches, lighters, thread, buttons, snaps, pins, make-up, hair care products, hair styling tools, nail care products, facial care products, body care products, perfume, and so forth);
hunting and gathering tools (such as fishing gear, bows and arrows, guns and ammunition, and so forth);
food preparation and serving items (such as cookware, bakeware, a can opener, eating utensils, cutlery, dinnerware, glassware, a coffee maker, a blender, a juicer, a food processor, a refrigeration device, a freezing device, a heat source including but not limited to a cook top, an oven, a gas-fueled or charcoal grill, fire starting aids, and so forth);
water purification apparatus (such as a filter, and so forth);
a personal communication device (such as one-way communication facilities, two-way communication facilities, a wireless communication device, and so forth);
a personal hygiene item (such as soap, a wash cloth, towels, shampoo, conditioner, a razor, a shaver, tissues, cotton balls, sanitary products, cotton swabs, contact lens solution, dental care products including, but not limited to a tooth brush, floss, mouth wash, tooth paste, and so forth);
healthcare products (such as vitamins, supplements, a hearing-aid, glasses such as prescription glasses, sunglasses, or reading glasses, contact lenses, prescription medicine, non-prescription medicine, a blood-pressure monitor, a thermometer, a first-aid kit, a walking cane, a walker, incontinence supplies, crutches, a wheel chair, a limb brace, a pacemaker, a stent, a structural implant, birth control product, sunscreen, removable and non-removable dental implants including, but not limited to dentures, a bridge, a retainer, and so forth);
non-human nourishment (such as dog food, cat food, bird food, and so forth);
barter medium (such as bulk or coined precious metals including but not limited to gold, silver, and other desirable materials, and so forth);
fuel supplies (such as gasoline, kerosene, diesel, ethanol, natural gas, propane, charcoal, flammable gels, and so forth);
power generation equipment (such as an electrical energy source including alternating current power sources, direct current power sources, fuel-consumptive power sources, renewable fuel source power sources, solar cells, generators, and so forth);
an entertainment apparatus (such as a media console, a media display, media accessories such as a DVD player and/or a VHS player and DVDs and/or VHS videos, a videogame console, videogames, game stations, videogame accessories, an audio player, headphones, a camera, a video recorder, a board game, playing cards, card game accessories, dice, chips, jacks, string, a yo-yo, a wheeled vehicle, a ball, coins, reading material such as teaching, school, educational or curriculum related material, a hardback book, a paperback book, a particular periodical such as a magazine, a newspaper, a newsweekly, other periodical publications such as a scientific journal, an artistic journal, an academic journal, or a special interest journal, a manual, reference material such as, an encyclopedia or a dictionary, a pamphlet, a review, a book on tape, a book on CD, a comic book, papers, literature, fiction, non-fiction, historical, memoirs, drama, poetry, prose, ballad, epic, legend, myth, folklore, essay, and so forth;
a musical instrument, gambling equipment, gambling accessories, racing equipment including, but not limited to remote control cars, and racing accessories, and sports equipment including, but not limited to balls for various ball games, sports gloves, nets, a bat, a Frisbee, and so forth);
physical conditioning, exercise, and maintenance, training and equipment (such as a yoga mat, jump rope, resistance bands, dumb bells and other free weights, tread mill, medicine balls, and so forth);
bedding, (such as mattresses of various kinds, blankets, pillows, sheets of various materials such as cotton, silk, satin, down, and/or synthetic fiber in any of a wide variety of thread counts and/or colors or patterns, towels, fabric supplies, and so forth);
repair and maintenance tools (hammer, nails, screw driver, screws, saw, tape measure, glue, tape, level, and so forth);
shelter supplies (such as lumber including, but not limited to framing lumber and plywood sheets, nails and/or other securement mechanisms, shingles, concrete, tools, or tarps, as well as stand-alone shelters such as tents and other portable shelters, and so forth);
child care supplies (diapers, bottles, blankets, formula, cribs, playpens, a pacifier, and so forth);
navigation equipment (such as a global positioning system (GPS) device, a compass, a map, and so forth);
educational tools (including but not limited to instructional texts, reference works, laboratory and demonstrative equipment, and so forth);
computer(s) (such as a lap top, hand held computer of personal digital assistant (PDA), and so forth);
personal effects (such as photographs, memorabilia, jewelry, a writing instrument, heirlooms, engraved objects, and so forth);
crafts supplies (such as yarn, knitting needles, fabric, needles, paper, paint, paint brushes, pencils, silk flowers, and so forth);
furniture (such as a mattress, a bed frame, desk, chairs, table, couch, and so forth); transportation vehicle(s) (such as an air-borne vehicle, a terrestrial vehicle, a water-borne vehicle, a hovercraft, an amphibious vehicle, a pilotless vehicle, a remotely piloted vehicle, and so forth);
related parts, accessories, and supplies (such as power cords, service parts to any of the other items included, and so forth)
   to illustrate but a few examples. It should be noted that some of the items included in the non-necessity listing may also be considered life-sustaining resources, especially under different circumstances. What constitutes a life-sustaining resource may vary depending on particular imminent threats, conditions, and the resources available. What one person may need to keep them alive another may find superfluous.

The additional non-necessity resources may also be customized based on categories, qualities, types, or quantities of resources. The non-necessity resources, like the life-sustaining resources can vary by gender, age, and so forth. By one approach, for example, a set of male-specific toiletries and/or male-specific clothing comprises male-specific resources while the female-specific resources can comprise female-specific toiletries and/or female-specific clothing. In addition, individuals can pick and choose items to be added to their bundle of resources. For example, one individual may chose to have access to reading glasses and various food preparation tools, while another individual may be interested in having access to specific articles of clothing and craft supplies. In addition to choosing the resource to receive, authorized beneficiaries may, if desired, chose the brand of a particular resource, such that the additional non-necessity resources may be upgraded from generic to luxury, designer, or brand name items. To illustrate, an authorized beneficiary who has chosen make-up to be included as a non-necessity resource may prefer a particular brand such as an Estee Lauder lipstick, a Lancôme foundation, or a Shiseido eye shadow.

Some authorized beneficiaries may chose to customize their resources relative to what other authorized beneficiaries have chosen. The authorized beneficiary may chose a group of people with whom resources may be combined together. For example, an extended family may only need one stove top; therefore, an authorized beneficiary may request a stove top provided no other authorized beneficiary in the group has already requested one. This allows groups of authorized beneficiaries and subscribers to coordinate their efforts to prepare for civilly-catastrophic events. In addition, authorized beneficiaries may request additional items if others have specifically not requested them. For example, one authorized beneficiary may request a certain number of batteries and may further request that number be doubled if no other authorized beneficiary in their group requests access to such batteries. Such customization does require information of the choices of other authorized beneficiaries.

As suggested above, a seemingly endless list of items are available to fulfill life's daily nutritional requirements, to say nothing of the huge number of requests that an authorized beneficiary may make with regard to non-necessities. Therefore, the process 100 also provides for establishing 105 a personal unit profile to track the myriad of possible requests made by the authorized beneficiaries and subscribers along with other characterizing information. The personal unit profile may store personal information such as the authorized beneficiary's name, age, gender, and address. Some resource customization may be accomplished purely based on this information. As another example, the profile may include information on whether the authorized beneficiary has any food allergies or intolerances. This information may also include data regarding whether an authorized beneficiary is a vegetarian or vegan and what foods the beneficiary will consume. In addition, the profile may include other details about the authorized beneficiary's food choices and preferences. If medical supplies are among the resources requested, additional information such as prescription and dosage information, blood type, medical conditions, and so forth may be useful or even required. Tracking this information can facilitate ensuring that when an authorized beneficiary requires certain resources that they have requested, that those resources are indeed available.

Such a catalog of information may also include, if desired, information regarding the authorized beneficiary's non-necessity resources. Not only may the name of the requested item be stored, but other specifics may also be included. For example, the information stored may include information regarding types of clothing, the size, the fabric, the cut, the color, the brand, and so forth. For each resource requested by the authorized beneficiary a number of pieces of information may usefully be stored in this regard.

This process 100 also accommodates acquiring or procuring 109 the customizable resources. Such procurement can be achieved through any of a variety of means. By one approach the items may be acquired on the open market. By another approach the items may be purchased or otherwise acquired from third parties via private negotiations. By yet another approach the entity that provides and accepts these subscriptions may itself create (through manufacturing, farming, or the like) the items of interest. In some cases, the acquired item may comprise a staple of ordinary commerce. In other cases, the acquired item may be unique and/or propriety to the acquiring/storing entity.

The process 100 also optionally provides for maintaining 111 the customizable resources pending a need to permit subscription-based access to the resources. This can comprise, for example, maintaining the resource on behalf of the authorized beneficiary and/or subscriber. The specifics of such maintenance will of course vary with respect to the nature of the resource or resources being maintained and also preferences of the authorized beneficiary and/or subscriber. The resources may be stored in a unit or bundle such that when access is desired to the resources, the resources are grouped together. Maintaining the resources may comprise ensuring the utility of the stored resources. The resources may need to be updated on a periodic basis to ensure, for example, freshness, usability, and efficacy of the resources. In one form, this can comprise monitoring the usability of perishable items and replacing such items on a corresponding schedule. As another simple illustration this step can comprise holding certain items in deep refrigeration or in other special storage conditions as may suit the proper maintenance of such items.

Such maintenance can also optionally comprise making adjustments to such supplies to reflect dynamically changing circumstances as occur during the consideration-based private civil security subscription period. Advancements in technology will often produce more efficient and effective resources, such that the previously stored resources will need to be substituted. As one illustration, a new item may become available that is particularly useful in dealing with or otherwise surmounting some condition that may likely arise upon the occurrence of a particular kind of civilly-catastrophic event. In such a case, maintaining such supplies can readily accommodate updating the acquired and stored items to include a supply of this new item.

Accordingly, such maintenance can readily comprise one or more of removing a particular one of the stored life-sustaining resources or non-necessities (as when a better substitute becomes available, when the stored resource is shown to be less effective for its intended purpose that was originally though, when the authorized beneficiary has chosen to receive another item, and so forth), adding additional ones of the stored life-sustaining resource or non-necessity (as when it becomes subsequently understood that more of a particular item is desired to achieve a particular survival related goal or purpose, or an authorized beneficiary has changed his preference), adding at least one new stored life-sustaining resource or non-necessity that is not already stored (as illustrated in the example provided above) and so forth.

As shown in FIG. 1, the personal unit profile may be updated 113 periodically to reflect changing circumstances. Over time, the needs and preferences of the authorized beneficiary will likely change. For example, an authorized beneficiary may outgrow clothing or have read reading material that was previously procured and stored such that new items need to be procured. In addition, the most likely catastrophe facing an authorized beneficiary at any given time or place may also change. Therefore, the personal unit profile may be changed to reflect the current needs, preferences, and situation of the authorized beneficiary.

After the profile is updated, the resources procured 109 and maintained 111 can be adjusted to reflect the updated profile. As suggested above, maintenance of the resource can include substituting certain resources when better options become available and thus, as circumstances change, different resources will likely be better able to protect and provide for the authorized beneficiary.

The process 100 also accommodates storing 115 the resources according to the authorized beneficiary and/or subscriber instructions. By one approach, the items may be stored at storage facilities that are owned or controlled by the subscription accepting entity. Any suitable storage facility may be used for this purpose with examples comprising (but not limited to) above ground man-made shelters, below ground or below water facilities (including purposefully excavated facilities, natural caverns, and so forth), and water borne facilities (such as barges and the like). By one approach such resources are stored in the aggregate. By another approach, an authorized beneficiary may choose a specific personal location for resource storage. By yet another approach, or in combination with an aggregated approach, some or all of the acquired resources are stored in subscriber-based bundles. For example, each such bundle can comprise a one year supply of basic life-sustaining resources for a single adult authorized beneficiary in addition to the specified non-necessity resources the beneficiary has requested. As another example, such a bundle may comprise a one year supply of life-sustaining resources as are intended for a family of four authorized beneficiaries. The use of bundles segregates at least some of the life-sustaining resources that correspond to an authorized beneficiary to form a unit that can be used when delivering the life-sustaining resources to that authorized beneficiary in response to a civilly-catastrophic event. While each storage facility may store resources corresponding to a plurality of authorized beneficiaries, an authorized beneficiary may chose to have their resources stored in a certain manner and/or at a certain location.

Other possibilities exist. For example, a single authorized beneficiary may have a first bundle comprised of staple items that are acquired and stored for all beneficiaries and a second bundle comprised of custom items that are specifically and uniquely acquired for this particular beneficiary or class of beneficiaries (such as beneficiaries who share a common dietary ailment or condition, sensitivity, medical condition, preference, or the like). In yet another embodiment, a plurality of bundles filled with specific staple supplies may be created such that a number of these staple bundles are aggregated to create a customized bundle. Both approaches provide for the formation of the resources into a plurality of discrete deliverable sub-units, wherein at least one of the sub-units comprises a quantity and selection of survival supplies as comprehensively correspond to the survival needs of the authorized beneficiary for a particular corresponding period of usage time. (As used herein, “comprehensively” will be understood to refer to a range of life-sustaining resources and non-necessity resources as will meet a corresponding range of anticipated needs.)

The bundle itself can be realized via any of a wide variety of encapsulating or restraining mechanisms. By one approach, at least some of these sub-units may comprise one or more pallets (as are known in the art) that have resources stacked thereon. For example, various tie-downs and/or tarps or other coverings can be employed in such a case to hold the resources in one place on the pallet. Palletization, in turn, will permit ready and efficient movement of such bundles to, about, or from a given storage facility and/or delivery of such a bundle to an authorized beneficiary when such delivery comprises the desired form of granting access to such resources. By another approach, at least some of the sub-units may comprise an open top (or side) or closable container made of wood, metal plastic, canvas, or other material of choice. Shrink wrap materials could also be employed in this regard. If the container is closable, if desired, the container may further be configured as a lockable container. In such a case, the sub-unit could be delivered in a locked state and would be unlocked, presumably, by the intended authorized beneficiary deliveree.

The use of such bundles (and particularly the bundling of staple items in a manner calculated to provide at least a minimum level of survival support to a predetermined number of authorized beneficiaries for at least a predetermined period of time) provides highly leveragable resource allocation opportunities as well. Such bundles also allow the subscriber to easily access additional items if the subscriber so chooses. It may be desirable for the authorized beneficiary and/or subscriber to have access to additional items post-catastrophe and having a plurality of extra bundles containing staple items may easily fulfill meeting that need.

If desired, this process can also optionally provide for access 119 to the resource in a manner chosen by the authorized beneficiary and/or subscriber. There are a number of customizable access options. By one approach, these teachings can be practiced by merely providing the supplies and resources such that gaining physical access to the resources comprises the responsibility of the authorized beneficiary. By one approach, the resources may be stored at a location and/or in a manner chosen by the authorized beneficiary and/or subscriber. For example, a rally point relatively close to an authorized beneficiary may be chosen.

In addition, as it is expected that catastrophic events will bring troubling and confusing circumstances, the resources may be accessed by the authorized beneficiary in other manners such as by delivery or drop off. It is contemplated that the drop off may occur at a predetermined location such as the home of the authorized beneficiary. Alternatively, the authorized beneficiary may chose to have the drop off location be the current location of the authorized beneficiary, whether or not the beneficiary is at the predetermined location. Access to the resources may or may not be prompted by a requested from the authorized beneficiary. For example, the resources may be delivered directly to the authorized beneficiary upon the occurrence of a civilly-catastrophic event without the authorized beneficiary making a request for access. By another approach, a request for the resources may be needed to precipitate delivery of or access to the resources.

It will be appreciated that these teaching provide for a highly flexible yet powerfully effective way by which a modern citizen can greatly improve their likelihood of surviving a civilly-catastrophic event. These teachings are sufficiently flexible so as to accommodate the needs and desires of a wide-ranging set of potential beneficiaries while nevertheless still tending to ensure adequate access to the basic necessities of life. Though training and some related activity may be provided and encouraged, in general the beneficiary receives those benefits without being required to make the commitment of time, energy, and expertise that would ordinarily be associated with attaining such a high level of civil security.

Those skilled in the art will recognize that a wide variety of modifications, alterations, and combinations can be made with respect to the above described embodiments without departing from the spirit and scope of the invention, and that such modifications, alterations, and combinations are to be viewed as being within the ambit of the inventive concept.

1. A method comprising:
accepting pre-catastrophe consideration-based private civil security subscription from a subscriber with respect to providing civilly-catastrophic event-based access to at least one life-sustaining resource to an authorized beneficiary;
customizing the at least one life-sustaining resource based on characterizing attributes of the authorized beneficiary.

2. The method of claim 1 wherein the at least one life-sustaining resource comprises at least one of:
hydration supplies;
nourishment supplies;
medical supplies;
environmentally borne threat abatement supplies.

3. The method of claim 1 further comprising establishing a personal profile for the authorized beneficiary using the characterizing attributes of the authorized beneficiary.

4. The method of claim 1 wherein the characterizing attributes comprise at least one of:
gender;
age;
dietary needs;
medical needs;
clothing needs;
physical needs;
personal preference.

5. The method of claim 1 further comprising procuring and maintaining the life-sustaining resource.

6. The method of claim 1 wherein the at least one life-sustaining resource comprises a plurality of life-sustaining resources.

7. The method of claim 1 further comprising providing an additional non-necessity resource.

8. A method comprising:
accepting pre-catastrophe consideration-based private civil security subscription from a subscriber with respect to providing civilly-catastrophic event-based access to at least one life-sustaining resource to an authorized beneficiary;
customizing the at least one life-sustaining resource based on preferences of the authorized beneficiary.

9. The method of claim 8 further comprising establishing a personal unit profile for the authorized beneficiary that catalogs the preferences of the authorized beneficiary with respect to the at least one life-sustaining resource.

10. The method of claim 9 further comprising procuring the life-sustaining resource to be received by the authorized beneficiary in accordance with the personal unit profile.

11. The method of claim 10 further comprising maintaining the life-sustaining resource to be received by the authorized beneficiary in accordance with the personal unit profile.

12. The method of claim 8 wherein the at least one life-sustaining resource comprises at least one of:
a consumable necessity of human life;
a non-consumable necessity of human life.

13. The method of claim 8 wherein the at least one life-sustaining resource comprises a plurality of life-sustaining resources.

14. The method of claim 12 wherein the consumable necessity of human life comprises at least one of:
hydration supplies;
nourishment supplies;
medical supplies.

15. The method of claim 14 wherein the hydration supplies comprise at least one of:
non-luxury hydration supplies;
luxury hydration supplies.

16. The method of claim 14 wherein the hydration supplies comprise at least one of:
generic hydration supplies;
brand-name hydration supplies.

17. The method of claim 14 wherein the nourishment supplies comprise at least one of:
non-luxury nourishment supplies;
luxury nourishment supplies.

18. The method of claim 14 wherein the nourishment supplies comprise at least one of:
generic nourishment supplies;
brand-name nourishment supplies.

19. The method of claim 14 wherein the consumable medical supplies comprise at least one of:
non-luxury medical supplies;
luxury medical supplies.

20. The method of claim 14 wherein the consumable medical supplies comprise at least one of:
generic medical supplies;
brand-name medical supplies.

21. The method of claim 12 wherein the non-consumable necessity of human life comprises at least one of:
medical supplies;
environmentally borne threat abatement supplies;
personal protection supplies.

22. The method of claim 21 wherein the non-consumable medical supplies comprise at least one of:
non-luxury medical supplies;
luxury medical supplies.

23. The method of claim 21 wherein the non-consumable medical supplies comprise at least one of:
generic medical supplies;
brand-name medical supplies.

24. The method of claim 21 wherein the environmentally borne threat abatement supplies comprise at least one of:
generic environmentally born threat abatement supplies;
brand-name environmentally borne threat abatement supplies.

25. The method of claim 21 wherein the environmentally borne threat abatement supplies comprise at least one of:
non-luxury environmentally borne threat abatement supplies;
luxury environmentally borne threat abatement supplies.

26. The method of claim 21 wherein the personal protection supplies comprise at least one of:
a face mask;
a gas mask;
gloves;
foul-weather gear;
head gear.

27. The method of claim 8 further comprising providing at least one additional non-necessity resource available upon a civilly-catastrophic event wherein an authorized beneficiary is able to customize the at least one additional non-necessity resource to be received.

28. The method of claim 27 wherein the at least one additional non-necessity resource comprises a plurality of additional non-necessity resources.

29. The method of claim 27 further comprising customizing the at least one additional non-necessity resource based on the preferences of the authorized beneficiary.

30. The method of claim 27 wherein providing at least one additional non-necessity resource comprises providing at least one of:
clothing;
sundries;
hunting and gathering tools;
food preparation and serving items;
water purification apparatus;
a personal communication device;
a personal hygiene item;
healthcare products;
non-human nourishment;
barter medium;
fuel supplies;
power generation equipment;
an entertainment apparatus;
physical conditioning, exercise, and maintenance training and equipment;
bedding, towels, and fabric supplies;
repair and maintenance tools;
shelter supplies;
child care supplies;
navigation equipment;
educational tools;
computer;
personal effects;
crafts supplies;
furniture;
transportation vehicle;
related parts, accessories, and supplies.

31. The method of claim 30 wherein the at least one additional non-necessity resource comprises at least one of:
a generic additional non-necessity resource;
a brand-name additional non-necessity resource;
a designer additional non-necessity resource.

32. The method of claim 31 wherein the clothing comprises at least one of:
a coat;
a jacket;
a poncho;
a top;
pants;
trousers;
jeans;
shorts;
a belt;
undergarments;
socks;
footwear;
cold-weather wear;
a skirt;
a dress
swimwear;
head gear.

33. The method of claim 31 wherein the sundries comprise at least one of:
batteries
flash lights;
threads;
buttons;
snaps;
pins;
make-up;
hair care products;
hair styling tools;
nail care products;
facial care products;
body care products.

34. The method of claim 31 wherein the food preparation items comprises at least one of:
cookware;
bakeware;
a can opener;
a heat source;
eating utensils;
cutlery;
dinnerware;
glassware;
a coffee maker;
a blender
a juicer;
a food processor;
refrigeration device;
freezing device.

35. The method of claim 31 wherein the personal communication device comprises at least one of:
one-way communication facilities;
two-way communication facilities;
a wireless communication device.

36. The method of claim 31 wherein the personal hygiene item comprises at least one of:
soap;
a dental care product;
a wash cloth;
towels;
shampoo;
conditioner;
a razor;
a shaver;
tissues;
sanitary products;
cotton swabs;
contact lens solution.

37. The method of claim 31 wherein the healthcare products comprise at least one of:
vitamins;
a hearing-aid;
glasses;
contact lenses;
prescription medicine;
non-prescription medicine;
at least one of removable and non-removable dental implants;
a blood-pressure monitor;
a thermometer;
a first-aid kit;
a walking cane;
a walker;
incontinence supplies;
crutches;
wheel chair;
limb brace;
pacemaker;
stent;
a structural implant;
birth control product;
sunscreen.

38. The method of claim 37 wherein the glasses comprise at least one of:
prescription glasses;
sunglasses;
reading glasses.

39. The method of claim 31 wherein the entertainment apparatus comprises at least one of:
a media console;
a media display;
media accessories;
a videogame console;
videogames;
videogame accessories;
an audio player;
headphones;
a camera;
a video recorder;
a board game;
playing cards;
card game accessories;
dice;
chips;
jacks
string;
a yo-yo;
a wheeled vehicle;
a ball;
coins;
sports equipment;
reading material;
a musical instrument;
gambling equipment;
gambling accessories;
racing equipment (remote control cars);
racing accessories.

40. The method of claim 39, wherein the reading material can comprise one of:
hardback book;
paperback book;
periodical;
magazine;
newspaper;
newsweekly;
periodical publications;
scientific journal;
artistic journal;
academic journal;
special interest journal;
manual;
educational text;
reference material;
encyclopedia;
dictionary;
pamphlet;
review;
book on tape;
book on CD;
comic book;
papers;
literature;
fiction;
non-fiction;
historical;
memoirs;
drama;
poetry;
prose;
ballad;
epic;
legend;
myth;
folklore;
essay.

41. The method of claim 31, wherein the personal effects comprise at least one of:
photographs;
memorabilia;
jewelry;
a writing instrument;
an heirloom;
engraved objects.

42. The method of claim 31 further comprising updating the personal unit profile of according to the preferences of the authorized beneficiary.

43. The method of claim 42 further comprising maintaining the at least one life-sustaining resource and additional non-necessity resource in accordance with the updated personal unit profile.

44. The method of claim 8, further comprising providing the subscriber with an option to acquire an additional life-sustaining resource.

45. The method of claim 44, further comprising providing the subscriber with an option to acquire an additional non-necessity resource.

46. The method of claim 8, further comprising maintaining the resources by at least one of:
in aggregation with resources for other authorized beneficiaries;
individually such that at least substantially all of the resources for one authorized beneficiary are bundled into a unit.

47. The method of claim 8 further comprising storing at least substantially all resources as have been customized with respect to the preferences for a given authorized beneficiary in a substantially bundled form factor that is separate and discrete with respect to resources as are stored for other entities.

48. The method of claim 8 further comprising storing the at least one life-sustaining resource at a location chosen by the subscriber or authorized beneficiary.

49. The method of claim 8 further comprising providing access to the at least one life-sustaining resource in a manner chosen by the subscriber or authorized beneficiary.

50. A method comprising:
accepting pre-catastrophe consideration-based private civil security subscription from a subscriber with respect to providing civilly-catastrophic event-based access to at least one life-sustaining resource to an authorized beneficiary;
customizing the at least one life-sustaining resource based on needs and requests of the authorized beneficiary.

51. The method of claim 50 wherein the needs are determined based upon at least one of:
gender;
age;
dietary needs;
medical needs;
clothing needs;
physical needs;
personal preference.

52. The method of claim 51 wherein the preferences comprise at least one of:
dietary preferences;
medical preferences;
clothing preferences;
physical preferences.

53. A method comprising:
accepting pre-catastrophe consideration-based private civil security subscription from a subscriber with respect to providing civilly-catastrophic event-based access to at least one life-sustaining resource to an authorized beneficiary;
customizing the at least one life-sustaining resource based on the requests of the subscriber.
